(19) United States
(12) Patent Application Publication
(10) Pub. No.: US 2013/0150714 A1
(43) Pub. Date: Jun. 13, 2013

(54) Vascular acess device ultrasound guidance system

(71) Applicants: Michael W. Howlett, Las Vegas, NV (US); James Victor Mercer, West Jordan, UT (US)

(72) Inventors: Michael W. Howlett, Las Vegas, NV (US); James Victor Mercer, West Jordan, UT (US)

(21) Appl. No.: 13/694,487

(22) Filed: Dec. 6, 2012

Related U.S. Application Data
(60) Provisional application No. 61/630,491, filed on Dec. 13, 2011.

(51) Int. Cl. A61B 8/00

(52) U.S. Cl. CPC A61B 8/4483
USPC 600/439

(57) ABSTRACT

A vascular access device ultrasound guidance system which provides a novel ultrasound imaging head interface for vessel access. The system comprises a fitted basket/VAD guide for attachment to a hand held ultrasound transducer. The VAD guide, being integrally molded to the fitted basket, comprises a releasible guide assembly for a VAD needle guide. In addition, the needle guide assembly comprises means for anchoring the VAD to a patient's skin following successful insertion. A magnetic needle guide system is disclosed. A fluid sampling device which integrates needle guidance and, safety needle capture in a single guide head is also disclosed.

### CONTINUATION-IN-PART

This U.S. Patent application is a Continuation-in-Part of a U.S. Provisional Application Ser. No. 61/630,491 titled VASCULAR ACCESS ULTRASOUND GUIDANCE SYSTEM (VADUGS) and filed Dec. 13, 2011.

### FIELD OF INVENTION

This instant invention is directly related to ultrasound imaging systems and associated devices which are employed in aiding needle access to veins and arteries.

### BACKGROUND AND RELATED ART

Use of ultrasound imaging is well known in medical art. Ultrasound guided vascular access devices (VAD) are being used with increasing frequency, particularly in patients with difficult-to-access veins and arteries. Use of ultrasound guidance, including using a needle guide is the most safe, accurate and effective method for VAD insertion currently available and is proven to increase first-stick success and decrease complications such as infiltration, hematoma and nerve damage. Ultrasound needle guides have been in use for many years. A common example of such being the Bard Access Systems' SiteRite® needle guide, commonly used in ultrasonic placement of central venous catheters (CVC), especially peripherally inserted central catheters (PICC). However, current needle guides are designed primarily for use with full barrier procedures where masks, gowns and full bed drapes are employed. Sterile draping is expensive, time consuming and used almost exclusively by vascular access specialists. Current needle guides are discarded after achieving vascular access and another device is used to secure the VAD to a patient's skin.

Also, there is no device known to Applicants which is currently available for combining a needle guide with a catheter securement device as a single unit. Combining an easy-to-use, systematized, drape-free sterile needle guide and VAD securement feature in single, dual-function device would offer distinct advantages including: 1) lower cost; 2) easier use; 3) more rapid procedure; 4) result in less waste material; 5) promotion of sterile technique when draping is not used; 6) convenient use of a needle guide that will reduce VAD insertion complications; and 7) better stabilization and securement of inserted VADs immediately after insertion in order to prevent inadvertent movement and/or removal of a VAD.

### BRIEF SUMMARY AND OBJECTS OF THE INVENTION

In brief summary, this novel invention provides novel apparatus and methods for vascular access and VAD securement. The VADUGS is comprised of two primary components: 1) a basket-like fitted transducer cover which includes a needle guide holder; and 2) a needle guide device, referenced by the term “guide and anchor assembly”, disclosed in detail hereafter. For brevity and clarity, the transducer cover and needle holder unit is identified by the term “basket” hereafter.

The basket may be designed to fit or snap onto an ultrasound transducer head. The basket has a sealed opening in the bottom that provides a lens which enables the transducer to “see” through and into adjoining tissue. The basket and sealed opening operate much like current practice of using a sterile sheath to cover a transducer head during VAD guidance. The basket, being sterile, assures sterile insertion of a needle/cannula used therewith.

The basket also includes a needle guide holder. The holder may comprise as plurality of slots molded into the basket to assure the needle guide is releasibly affixed onto the basket at a pre-desired position for achieving a proper angle and depth for tissue entry of an associated needle/cannula. As such, the needle guide holder permits multiple angles of VAD insertion to meet an objective of more facile and accurate targeting of vessels at varying depths. As an example, one embodiment of the instant invention, provides three slots at varying angles to achieve the objective.

The guide and anchor assembly comprises a flexible and close fitting needle guide channel into which a needle/cannula is fitted. Further, the guide and anchor assembly has wings which are then fitted into the needle guide holder of the basket. For such, the needle guide holder may comprise slots for positioning an associated guide and anchor assembly channel and needle/cannula into a desired position and angle for advancing the needle/cannula through the skin and into a target vessel.

The guide and anchor assembly comprises three parts which may be constructed as a single unit: 1) a needle guide channel; 2) a “hub'lock” device into which a VAD hub can be releasibly affixed; and 3) a surface (preferably winged) having an applied adhesive for adhering to a patient's skin. The guide and anchor assembly is shaped and sized to securely attach to the needle guide holder. For example, the guide and anchor assembly may be so attached by a friction fit to assure no movement relative to the needle holder without substantial removal force, once attached.

### Method of Use

1. A predetermined ultrasound probe is affixed to the basket.
2. A desired slot in the basket is selected for target vessel depth.
3. The guide and anchor assembly is secured into the selected slot.
4. The VAD is loaded into the needle guide channel of the guide and anchor assembly.
5. The associated ultrasound probe, guide and anchor assembly and VAD are displaced to be over a target vessel.
6. Once so disposed as determined by ultrasound imaging, the VAD is advanced until the target vessel is cannulated.
7. Then the VAD is removed from the needle guide channel and the Basket/guide and anchor assembly, permitting the basket/guide and anchor assembly to be set aside.
8. The VAD is fully advanced and correct placement is confirmed.
9. The guide and anchor assembly is retrieved from a holder on the basket and secured to a hub on the VAD via a locking mechanism (such as a bayonet locking mechanism) to securely retain the VAD.
10, Adhesive upon the winged surface of the guide and anchor assembly is exposed by removing protective strips, permitting the guide and anchor assembly to be adhesively affixed to skin of the associated patient.

In another embodiment, a magnetic needle guide is fitted into the Basket in the place of the guide and anchor assembly. To provide for a variety of needle sizes, a V-shaped channel is disposed within the effective lines of force of a magnet used as part of the needle guide. Noting that there is only moderate force required to move the needle parallel to a magnet, a needle is easily displaced along the V-channel. For like reasons, a needle/cannula is easily removed by gentle linear displacement along the V-channel. As such, no integrated securement system, similar to that of the guide and anchor assembly is required for the magnetic needle guide.

Yet another embodiment which is associated with the instant invention is an integral needle block. The integral needle block also fits into the basket in the same manner as other needle guide devices. In this embodiment, the needle, typically used for a single vessel access as in blood sampling, is not removable from the needle guide. As such, the needle guide block performs as a needle lock-out safety device once desired material is obtained and the needle is no longer needed.

Accordingly, it is a primary object to provide a novel ultrasound needle guide and associated catheter securement unit.

It is an important object to provide an integrally molded transducer basket and needle guide holder which has a plurality of guide slots for achieving various angles and depths of penetration for accessing a vessel.

It is a chief object to provide a needle holder and guide for both providing a vascular access device guide and an anchor at a patient skin penetration site.

It is an object to provide a basket which can be mechanically affixed to a selected ultrasound transducer head.

It is another important object to provide a needle guide device which is securely affixed to a vascular access device for assured containment.

It is another object to provide a needle guide to which a needle is affixed by magnetic force.

It is yet another object to provide apparatus and method for applying gel to an ultrasound head and to an ultrasound communication site of a basket.

It is a basic object to provide a vascular access device ultrasound guidance system which provides a needle safety enclosure for a needle.

These and other objects and features of the present invention will be apparent from the detailed description taken with reference to accompanying drawings.

### BRIEF DESCRIPTION OF THE DRAWINGS

FIG. 1 is a perspective of a transducer basket and needle guide.

FIG. 2 is a perspective of a needle guide and anchor assembly made according to the instant invention and which may be used in conjunction with the transducer basket and needle guide seen in FIG. 1.

FIG. 3 is a perspective of the needle guide and anchor assembly seen in FIG. 2 disposed for use in the transducer basket and needle guide seen in FIG. 1.

FIG. 4 is a perspective of the needle guide and anchor assembly seen in FIG. 2 and a mode of insertion of a VAD into the needle guide and anchor assembly.

FIG. 5 is a perspective of the parts seen in FIG. 4 wherein the VAD is partially displaced into the guide.

FIG. 6 is a perspective of the parts seen in FIGS. 4 and 5 with the VAD fully inserted and locked in position and a backing strip partially removed from wings of the needle guide and anchor assembly for adhesing the needle guide anchor assembly to a patient's skin.

FIG. 7 is a perspective of the transducer basket and needle guide, needle guide and anchor assembly and VAD assembled preparatory to an insertion procedure.

FIG. 8 is a perspective of the transducer basket and needle guide seen in FIG. 1 with a schematic of an ultrasound transducer head disposed within the basket.

FIG. 9 is a perspective of a basket disposed within a gel dip basin displayed in schematic format, this basin having an adhesive strip affixed to the bottom.

FIG. 10 is a perspective of an exploded view of a magnet guide for the instant invention.

FIG. 11 is a perspective of an assembled magnet guide seen in FIG. 10 with a needle inserted and magnetically held therein.

FIG. 12 is a perspective of the magnet guide seen in FIG. 10 disposed in a transducer basket and needle guide seen in FIG. 1.

FIG. 13 is a perspective of an integral needle block guide with hidden line paths for needle insertion.

FIG. 14 is a perspective of the integral needle block guide with a VAD inserted through a path thereof.

FIG. 15 is a perspective of the combination seen in FIG. 15 with a needle of the VAD locked to prevent removal.

### DETAILED DESCRIPTION OF THE ILLUSTRATED EMBODIMENT

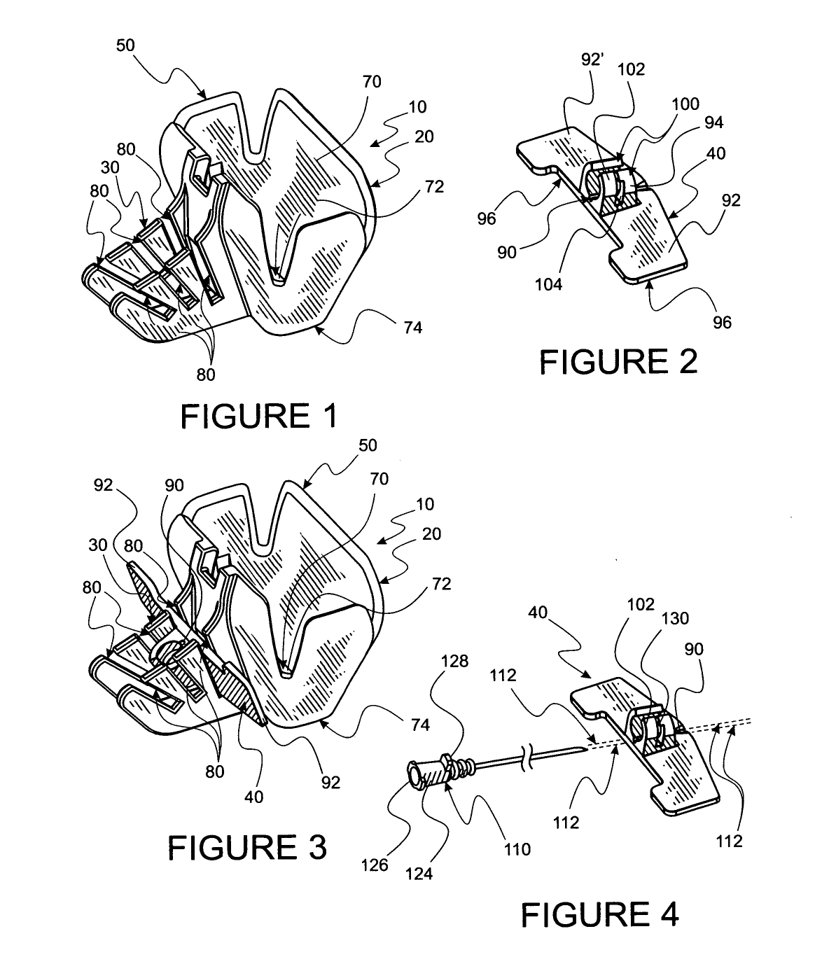

In this description, the term proximal is used to indicate the segment of the device normally closest to the object of the sentence describing its position. The term distal refers to the other end. Reference is now made to the embodiments illustrated in FIGS. 1-15 wherein like numerals are used to designate like parts throughout. Primes of numbers are used to identify parts being similar in form and function, but not identical to parts numbered without primes.

VADUGS 10, a portion of which is seen in FIG. 1, comprises two primary components, 1) a transducer basket 20 and a needle guide holder 30, and a second portion, seen by example in FIG. 2, comprising a needle guide device, an example of which is a guide and anchor assembly 40. Within the scope of the instant invention, a needle guide device may take a plurality of forms and functions. This example of such a needle guide device is guide and anchor assembly guide 40, disclosed in detail hereafter. As disclosed supra, an integral combination of the transducer basket 20 and needle guide holder 30 is further identified as basket 50.

### Basket 50

Transducer basket 20 is shaped and sized to conform to a predetermined ultrasound transducer head 60 (see FIG. 8) such that transducer basket 20 snugly fits or snaps onto ultrasound head 60, permitting basket 20 and head 60 to be used as a single unit. Basket 50 has a lens portion 70 which is formed to provide sealed containment for head 60 while being substantially transparent to ultrasound impulses emitted from head 60. Basket 20 and lens portion 70 perform in similar fashion to a sterile sheath used to cover a transducer head during VAD guidance in conventional ultrasound imaging procedures. Of course, any surface of basket 20 which contacts any part associated with skin penetration should be sterile.

Needle guide holder 30 seen by example in FIG. 1 is preferably integrally molded with transducer basket 20. Holder 20 comprises a plurality of slots (generally numbered 80) disposed at varying predetermined angles to achieve proper angles and depths of needle/cannula tissue entry. As such, needle guide holder 30 allows for multiple angles of VAD insertion to better target vessels at varying depths per arrangement of slots 80. The embodiment of slots 80, seen in FIG. 1, provides three slots of varying angles for needle guide device 40.

A variety of needle guides have been defined for use with basket 50. While the number is not limited to those disclosed herein, three such guides are hereafter disclosed in detail. They are: 1) a guide and anchor assembly; 2) magnetic needle guide; and 3) a guide and capture needle block.

### Guide and Anchor Assembly 40

Guide and anchor assembly 40 combines an ultrasound needle guide channel and a VAD securement feature in a single device. As seen in FIG. 2, guide and anchor assembly 40 comprises a needle guide channel 90 sized and shaped to provide controlled guidance for a needle/cannula of a VAD. When disposed within channel 90 a needle/cannula may be freely moved longitudinally within the channel without being dislodged, unless thrust out by undue force. A pair of opposing planar wings (generally numbered 92) are sized and shaped to snugly fit within guide slots 80. Note that so positioning wings 92 determines position and direction of channel 90.

Other than channel 90, two primary parts of guide and anchor assembly 40 are wings 92 and a hub-lock 94. Wings 92 not only provide for guidance when inserted into slots 80, but, being planar, are fitted with an adhesive on the under surface 96 thereof (see FIG. 6). By providing adhesive on underside 96 of wings 92, once a needle has been threaded into a target vessel, guide and anchor assembly 40 may be displaced from slots 80 and disposed as a butterfly adhesively affixed to a patients skin to construct an anchor for the associated VAD. Of course, prior to affixing under surface 96 to a patients skin to anchor guide and anchor assembly 40, the adhesion on under surface 96 must be protected. For such, a removable backing strip 98 should be provided as seen in FIG. 6.

Hub-lock 94 provides a hub securing member 100 for releasibly affixing a VAD hub to assembly 40. As an example, a bayonet-style locking member 102 is seen in FIG. 2. In summary, assembly 40 is designed for two modes of use: 1) to be securely affixed to a needle guide channel in an insertion mode and 2) to be securely affixed to a patients skin in an anchoring mode.

### Method of Use of Guide and Anchor Assembly 40

Following are a series of exemplary steps for using assembly 40:
1. Affix a transducer head 60 into transducer basket 20 (See FIG. 8). (Note: prior to inserting head 60 into basket 20, both sides (interior side 72 and exterior side 74, as seen in FIG. 3) of lens 70 should be coated with gel as prescribed by manufacturer of head 60 to assure clarity of ultrasound signal transmission.)
2. Choose a desired slot 80 for angle of entry and target vessel depth for a selected VAD. (See slot examples in FIG. 3.)
3. Insert an assembly 40 into the chosen slot 80. (See FIG. 3.)
4. Insert the selected VAD (generally 110) into guide channel 90 (See FIG. 4.)
5. Displace head 60 and lens 70 to a site above a target vessel.
6. Cannulate the target vessel by displacing VAD 110 inferiorly (See FIG. 7.).
7. Detach the VAD 110 from VADUGS 10 and assembly 40.
8. Fully advance VAD 110 and check for appropriate placement.
9. Retrieve assembly 40 (see FIG. 2).
10. Secure VAD 110 into hub-lock 94. (See FIG. 6)
11. Remove backing strip 98 from adhesive side of wings 92. (See FIG. 6) Note: Backing strip should not be removed from wings 92 until after VAD is secured into bayonet lock.
12. Anchor assembly 40 to patient skin (Not shown in figures as such is well known in catheter anchoring art.)

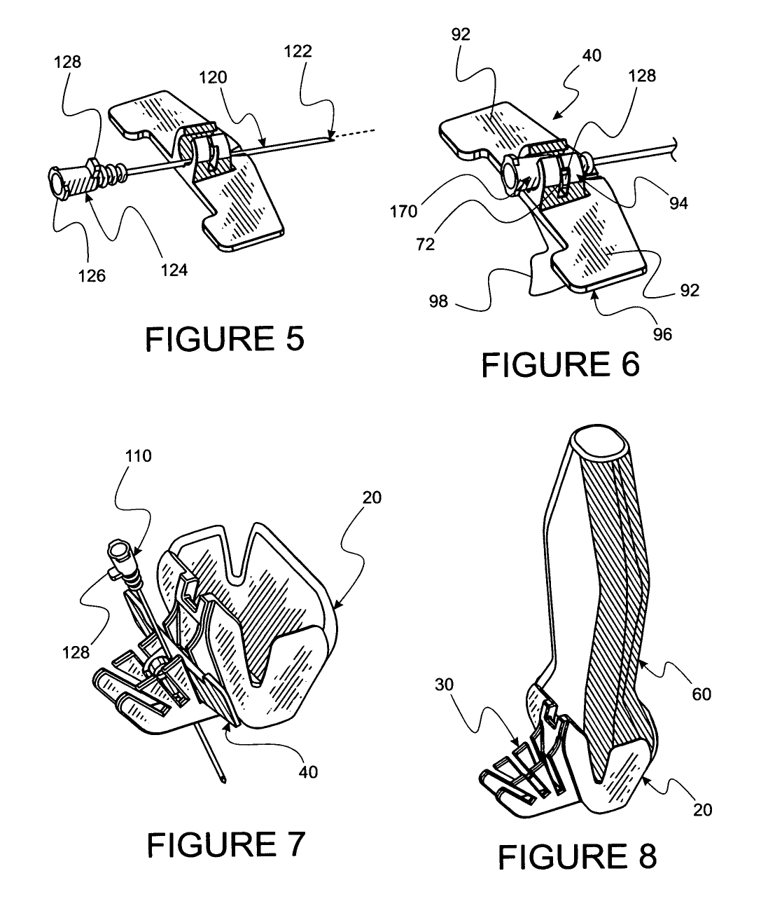

Reference is now made to FIGS. 4-6 wherein a VAD 110 is seen in various stages of displacement through a guide channel 90 into assembly 40. As seen in FIG. 4, a VAD 110 is displaced in direction of dashed lines 112 toward engagement in assembly 40. Once partially engaged, as seen in FIG. 5, a portion 120, having a sharpened entry end 122 protrudes distally from assembly 40 for ultimate skin entry.

A needle hub 124 generally comprises a luer fitting 126 for upstream fluid communication and an outwardly protruding locking member 128. Locking member 128 is sized and shaped to latch in bayonet fashion within slots 130 of locking member 102. Note: Affixing VAD 110 in slots 130 is performed only after VAD 110 is fully advanced and in appropriate placement within a target vessel. VAD 110 is so disposed in FIG. 6.

### Magnetic Needle Guide 40′

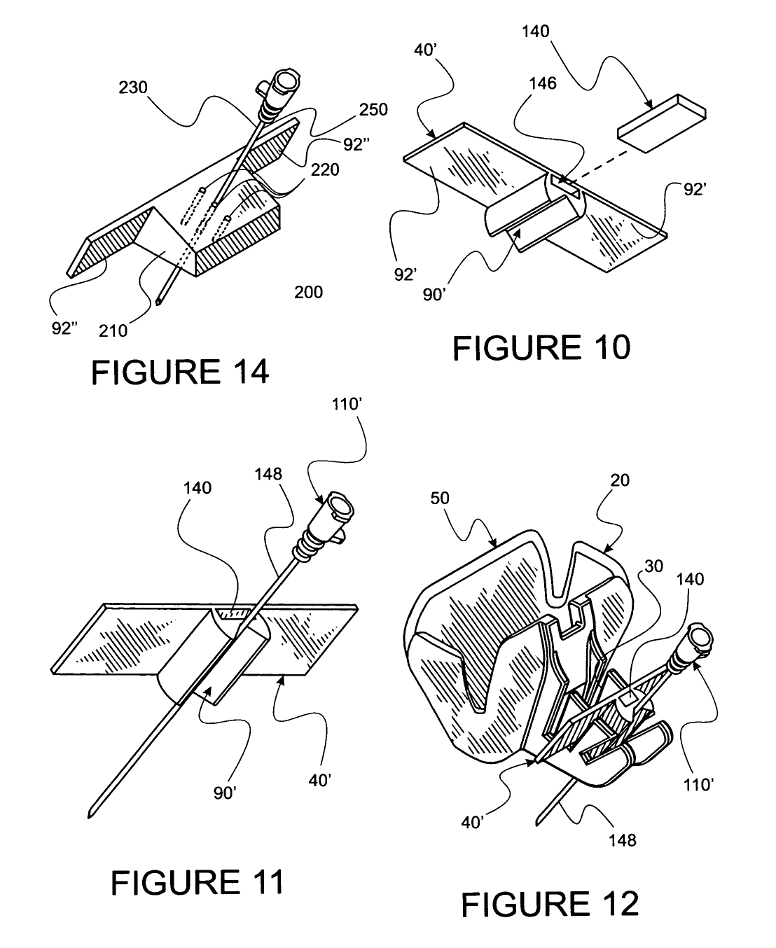

Reference is now made to FIGS. 10-12 wherein a magnetic needle guide 40′ is variously seen. As seen in FIG. 10, guide 40′ comprises a pair of wings, generally numbered 92′ and a “V” groove 90′ which acts as a guide for a VAD 110′. Guide 40′ also comprised an imbedded magnet 140 which is sized and shaped to be imbedded in an enclosing housing 142 in a centrally disposed guide element 144 which longitudinally disposed relative to V groove 90′. VAD 110′ is like VAD 110, except for a needle which is made of material which can be magnetized by magnet 140.

Magnet 140 (see FIG. 10) is seen inserted into slot 146 (see FIG. 10) in assembly 40′, as seen in FIG. 11. Form of V groove 90′ permits a magnetizable needle 148 of a VAD 110′ of varying size to be affixed by magnetic force thereat. VAD 110′ is seen disposed for penetration by needle 148 in FIG. 12.

### Guide and Capture Needle Block 200

A guide and capture needle block, generally numbered 200, is seen in various stages of use in FIGS. 13-15. Block 200 generally comprises a pair of wings 92″ and a block part 210 all of which are integrally molded to form a needle guide for use with VADUGS 10. Note that wings 92″ may perform the same skin securing function as wings 92 and 92′. Block part 210 comprises a plurality of through holes, generally numbered 220. Each hole 220 is varied in size from each of the other holes 220 in diameter to permit, by selection, an appropriate hole 220 sized for a selected VAD needle. Note that all holes 220 are preferably disposed to guide each selected needle to a common penetration site.

Further, for each VAD 110 used with block part 210, a specially formed needle 230 having a notch 240 circumferentially disposed about the exterior 250 of needle 230. Also, each hole 220 has a molded latch, commonly numbered 260, disposed therein. Such notches and latches are well known in the plastics molding art. It should be noted that latches 260 are formed to be permissive to displacement of needle 230 in a distal direction of insertion, but made to effectively latch against notch 240 when needle 230 is displaced proximally toward a user. Such provides a needle safety mechanism for needles after use.

### Gel Dip Basin

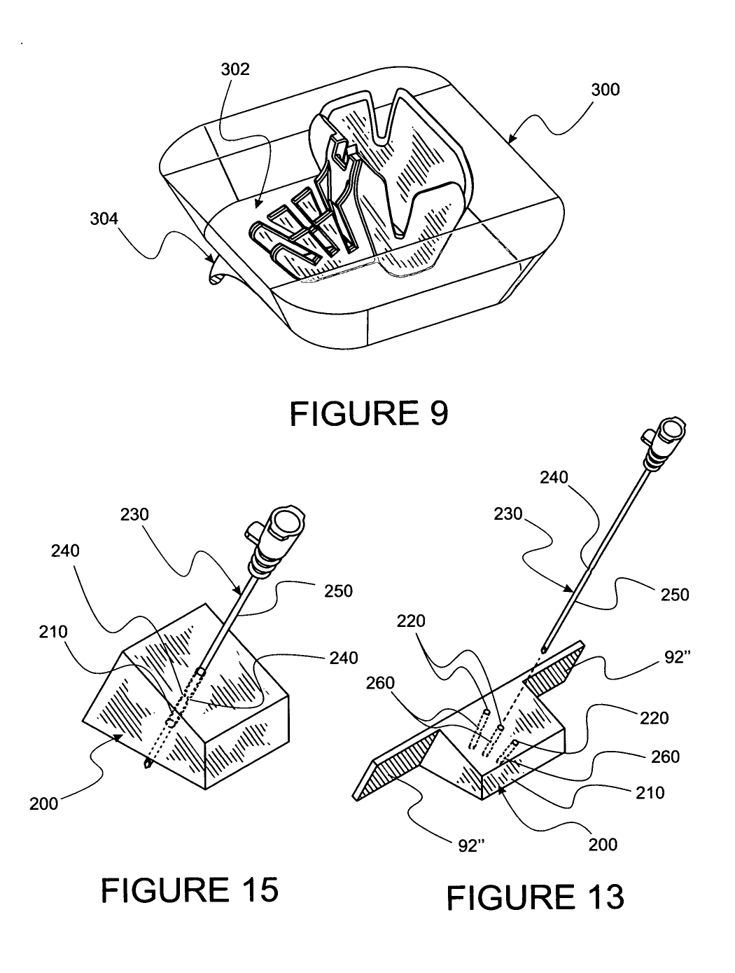

As seen in FIG. 9, associated with VADUGS 10 is a dip basin 300 for gel 302, (deposited therein) to be applied to a surface to facilitate ultrasound transmission. Such gels are commonly used in ultrasound imaging procedures. As sterility of gel is very important in such applications, it is anticipated that dip basin 300 is usually pre-molded, filled, sealed with a removable cover and sterilized before use.

As such, with a protective cover removed, a sterile transducer head 60 can be first dipped into gel provided within basin 300. Thereafter, head 60 can be inserted into basket 20, as seen in FIG. 8. Basket 50 is seen disposed in basin 300 to affix gel to lens 70 exterior side 74 in FIG. 9. Note that the above disclosed operation associated with basin 300 may be performed with a single hand, simplifying associated procedures and lessening risk of contamination if securely affixed to a work surface. For single handed operation, basin 300 may best be secured at a dipping site by adhesive applied to an underside. A backing 304 is seen partially removed in FIG. 9. Using adhesive protected by backing 304, basin 300 may be securely affixed to a planar surface prior to application of gel to either transducer head 60 or basket 50.

The inventions disclosed herein may be embodied in other specific forms without departing from the spirit or essential characteristics thereof. The present embodiments are therefore to be considered in all respects as illustrative and not restrictive, the scope of inventions being indicated by the appended claims rather than by the foregoing description, and all changes which come within the meaning and range of equivalency of the claims are therefore intended to be embraced therein.

What is claimed and desired to be secured by Letters Patent is:

1. A vascular access device ultrasound guidance system for imaging vessels by providing guidance for vascular access devices and for providing for vascular access device securement, said system comprising:
an integrally molded transducer basket and needle guide holder which comprises a plurality of guide slots for an associated needle guide device;
the needle guide device comprising a pair of wings and structure for affixing and guiding direction of insertion for a vascular access device;
said wings comprising sufficient rigidity, thickness and expanse to assure proper direction of needle insertion when disposed within said slots;
said wings further comprising sufficient flexibility and planarity for skin adhesion and an adhesive applied to contacting surfaces such that, when disposed upon a patients skin, said wings and associated needle guide provide an anchor for the associated vascular access device after vessel penetration.

2. A vascular access device ultrasound guidance system according to claim 1 wherein said basket comprises a molded form by which said basket is securely bur releasibly, mechanically affixed to a predetermined ultrasound head.

3. A vascular access device ultrasound guidance system according to claim 1 wherein said structure comprises a guidance channel which provides directional control for vascular access device insertion and containment when wings are affixed to a patients skin to thereby anchor the vascular access device after successful vascular access device insertion.

4. A vascular access device ultrasound guidance system according to claim 3 wherein said structure comprises a bayonet lock about the guidance channel for securing the associated vascular access device to the needle guide device.

5. A vascular access device ultrasound guidance system according to claim 3 wherein said structure comprises a magnet disposed to hold a needle which can be held in place by magnetic force within the channel.

6. A vascular access device ultrasound guidance system according to claim 1 wherein said wings comprise a removable cover placed over said adhesive for protection until removed for purposed of anchoring the associated vascular access device.

7. A vascular access device ultrasound guidance system according to claim 1 further comprising a dip basin for covering a lens of said basket for clarity of ultrasound transmission.

8. A vascular access device ultrasound guidance system for imaging vessels by providing guidance for needle based vascular access devices and for providing for needle securement, said system comprising:
an integrally molded transducer basket and needle guidance device which comprises a guide block comprising a portion having a predetermined thickness opposing direction of insertion of the vascular access device and at least one guide hole through the portion;
each at least one guide hole comprising a latch proximally disposed a predetermined distance from an associated exit orifice for the needle;
the needle comprising a sharpened end and an exterior diameter in which an annular notch is proximally disposed thereto;
said latch and said notch being disposed to permit distal
displacement of the vascular access device but to catch and hold the needle of the access device thereby providing a needle safety device.
